(19) United States
(12) Patent Application Publication
(10) Pub. No.: US 2023/0043035 A1
(43) Pub. Date: Feb. 9, 2023

(54) AGAROPHYTON CHILENSIS EXTRACT, RICH IN FREE FATTY ACIDS, AS A NUTRACEUTICAL OR NUTRITIONAL SUPPLEMENT, SUITABLE FOR MODULATING PPAR# ACTIVITY

(71) Applicants: UNIVERSIDAD ANDRES BELLO, Santiago (CL); PONTIFICIA UNIVERSIDAD CATÓLICA DE CHILE, Santiago (CL)

(72) Inventors: Francisca Verónica BRONFMAN CÁCERES, Santiago (CL); Karen Andrea FUENZALIDA MORALES, Santiago (CL); María Raquel IBÁÑEZ HORMAZABAL, Santiago (CL); Loretto Matilde CONTRERAS PORCIA, Santiago (CL); Víctor BARRAZA FUENTES, Santiago (CL)

(21) Appl. No.: 17/756,352

(22) PCT Filed: Nov. 22, 2019
(86) PCT No.: PCT/CL2019/050117

§ 371 Date: May 23, 2022

(51) Int. Cl. A61K 36/04; A61P 25/28; A61P 9/10; A61K 31/20

(52) U.S. Cl. CPC A61K 36/04; A61P 25/28; A61P 9/10; A61K 31/20; A61K 2236/00; A61K 2236/33

(57) ABSTRACT

Agarophyton chilensis extract, enriched with free fatty acids and PPARγ modulators, comprising palmitic acid, stearic acid, myristic acid, oleic acid, and 8-hydroxyeicosatetraenoic acid (8-HETE). Method for obtaining the extract. Nutraceutical composition, comprising the extract enriched with free fatty acids and PPARγ modulators, wherein said extract is useful for treating or preventing health problems, which require neuroprotection, wherein said neuroprotection involves activation of PPARγ receptors.

### CROSS-REFERENCE TO RELATED APPLICATIONS

The present application is a National Stage of International Application No. PCT/CL2019/050117 filed on Nov. 22, 2019, the entire contents of each hereby incorporated by reference.

### FIELD

The claimed disclosure refers to a naturally occurring extract enriched in free fatty acids and natural activators of the PPARgamma (PPARγ, Peroxisome Proliferator Activated Receptor gamma) transcription factor. Said extract has preventive, mitigation, or therapeutic effects on diseases with inflammatory components and/or metabolic diseases, such as type II diabetes, pulmonary hypertension and chronic and acute neurological diseases, such as neurodegenerative, and cerebrovascular diseases respectively. The presently claimed extract is useful in the treatment of all those diseases where PPARγ pharmacological activators are described as reducing damage or favoring the functional recovery of the organ, or the integrity of the tissue, as it is the case of the vasculature and nervous tissue. The enriched extract, among its main components, comprises palmitic acid and 8-HETE. In addition, the extract among its components comprises stearic acid, myristic acid, oleic acid, 9-hydroxyeicosatetraenoic acid (9-HETE), and further oxidized fatty acids derived from arachidonic acid (eicosanoids). The enriched extract could be used as a nutraceutical formulation or nutritional component.

In the claimed disclosure, said naturally occurring extract enriched in free fatty acids and natural activators of the PPARγ transcription factor is obtained by chromatographic techniques, which are detailed below.

### BACKGROUND

As indicated above, the disclosure is directed to natural extracts enriched, by means of chromatographic separation methods, with naturally occurring PPARγ activators and free fatty acids for use as a nutraceutical product and as a nutritional component (or ingredient) useful for preventing, mitigating or treating diseases where the use of PPARγ agonists would have therapeutic effects and wherein said claimed extract mainly comprises palmitic acid and 8-HETE; said extract also comprising further acids, such as stearic acid, myristic acid, oleic acid, 9-HETE, besides other eicosanoids at a lower ratio.

The PPARγ transcription factor belongs to the PPAR family together with PPARαlpha (PPARα) and PPARbeta/delta (PPARβ/δ), and it is known that its absence is lethal from embryonic development. PPARs are a family of ligand-induced transcription factors which belong, in turn, to the superfamily of nuclear hormone receptors. Collectively, they are lipid sensors and regulate insulin sensitivity, mitochondrial biogenesis, and glucose and lipids homeostasis. The activation of PPARs is reported to have anti-apoptotic, anti-oxidative, and anti-inflammatory activity. The function of these receptors is modified by the binding of synthetic and natural lipid-source ligands and by co-activator and co-repressor proteins, which may stimulate or inhibit the receptor function, respectively.

PPARγ has been postulated as a therapeutic target for different diseases with inflammatory and/or metabolic components; being thus involved in health problems, such as obesity, diabetes, atherosclerosis, and chronic and acute neurodegenerative diseases, since the use of PPARγ agonists decrease the adverse effects of these diseases on the physiology in different animal and human models. Their expression and activity have also been observed as necessary for the proper function of various tissues, including fatty tissue, vasculature, liver, muscle, and nervous tissue. The most tested PPARγ pharmacological agonists are thiazolidinediones (TZD), which include rosiglitazone, pioglitazone, and troglitazone. TZDs are synthetic drugs and were the first PPARγ ligands found. Even though several beneficial effects have been described for these agonists, in particular as insulin sensitizers and on the reduction in blood sugar, their use has been restricted for humans, given that they have adverse side effects, such as the increased risk of infarction, increased cardiac output, increased body weight, and peripheral edema. These side effects are considered to be associated with the PPARγ activation mechanism since they stabilize the PPARγ ligand binding site in a conventional manner, being in two states: on or off, and for this reason, the TZDs are named full-agonists. Functions, as a partial agonist or SPPARM (Selective PPAR Modulators) or both are other ways of activating the receptor. These kinds of molecules are more versatile ligands so that they can bind to the receptor-binding site in different manners, causing dynamic and slow conformational changes. Since PPARγ is a transcription factor that modifies the expression of several genes, recruiting various co-activators and co-repressors, the partial ligands and SPARMs have been reported as capable of inducing recruitment of some co-activators, but not others, resulting in different physiological responses. For example, some SPPARMs increase insulin sensitivity, but without adipogenic effects compared to those observed for TZDs. There is evidence indicating that SPPAMRs could be drugs suitable for the treatment of type II diabetes. Therefore, the search for natural or synthetic partial agonist and SPPARMs is still ongoing.

It has been described that activation of PPARγ can have several actions in cell physiology, including the aforementioned anti-inflammatory effects on several types of cells, such as glial cells in the brain and blood lymphocytes, which are cells whose activation contributes to the onset and progression of the damage that occurs in neurological diseases.

More recently, it has been acknowledged that PPARγ plays an essential role in immune response through its capability of inhibiting the expression of inflammatory cytokines and directing the differentiation of immune cells towards anti-inflammatory phenotypes.

Ischemic stroke (IS) is a serious worldwide health problem since there are no effective treatments for treating it or reducing its incidence in high-risk populations. To date, the only pharmacological treatment approved by the US FDA for stroke is the tissue plasminogen activator (tPA), which must be administered before 3-5 hours after onset of symptoms. However, the narrow time window and the risk of intracerebral hemorrhage constitute a hindrance to use it as an effective treatment. One of the technical problems addressed by the presently claimed disclosure relates to the provision of the formulation of a nutraceutical, a functional ingredient or nutritional component for use as neuroprotector, being capable of promoting recovery of lost motor skills, even in hemorrhagic strokes, providing broader time windows, and showing a mode of action different from that of tPA. The application scope of this nutraceutical would be broader than tPA, because besides covering patients who already had a stroke, it could further cover patients with risk factors, such as people who have suffered a transient ischemic attack or those who have already suffered a stroke. Moreover, hypertense people and people with type II diabetes resistant to conventional treatments could get benefit of the above mentioned treatments based in a functional ingredient.

The inventors have focused on the neuroprotective properties obtained with the activation of PPARγ since it has been demonstrated that the activation of this receptor mitigates the inflammation associated with chronic and acute neurological injuries.

Hence, the present disclosure provides an enriched extract that can be used as nutraceutical, functional ingredient or nutritional component with neuroprotective effects and with a low or no presence of side effects, being a naturally occurring extract that activates PPARγ without promoting adipogenesis. The extract can be provided as a nutraceutical, useful for preventing and posterior treatment of stroke. This nutraceutical is particularly useful as a preventive measure in populations with high-risk of suffering a stroke, such as people with arterial hypertension (AHT) and diabetes mellitus.

The possibility that some nutraceuticals can act as neuroprotective agents has been evaluated in the state of the art. However, usually, most nutraceuticals directed to only neuroprotection correspond to compounds with antioxidant capacity. For example, in the market, there are two nutraceuticals based on herbal extracts directed to neuroprotection against stroke. One of them being the Dan Shen agent, used in China, the main component of which is the root of Salviae miltiorrhizae (red sage), and the other nutraceutical being NeuroAID, which corresponds to a mixture of 9 natural extracts. Although their use is recommended for stroke, none of these are supported by exhaustive clinical trials and, furthermore, none of the said nutraceuticals have an effect or components related to PPARγ.

In the publication WO2014186913, the group of inventors of the presently claimed disclosure provided a process for obtaining an oleoresin from red algae, wherein the capability of this type of oleoresin to trigger PPARγ activation was first presented. This publication reports that the red algae, Agarophyton chilensis, subjected to stress, generates molecules which would correspond to endogenous ligands of the PPARγ nuclear receptor in mammals. In WO2014186913, the inventors discussed the disadvantages of using TZDs, compounds acting as PPARγ ligands, which modulate insulin resistance, having neuroprotective, neuro-regenerative and anti-inflammatory effects, but also having side effects, such as increased risk of coronary heart disease, peripheral edema and macular edema.

Mišurcová, L., et al. (Mišurcová, L., Ambrožová, J., & Samek, D. 2011. Seaweed lipids as nutraceuticals. (Advances in food and nutrition research, 64: 339-355. Academic Press) reports that seaweeds are known as a low energy food, wherein the polyunsaturated fatty acids omega-3 and omega-6 represent a significant portion of lipids in seaweeds, forming an essential part of all cell membranes and precursors of eicosanoids that are essential bioregulators of many cellular processes. Polyunsaturated fatty acids effectively reduce the risk of cardiovascular disease, cancer, osteoporosis and diabetes. The primary commercial sources of omega-3 polyunsaturated fatty acids are fish, but their typical fishy smell has limited their use as food additives, as well as their oxidative instability. However, the growing needs for healthy functional foods have led to the production of polyunsaturated fatty acids as nutraceuticals in a batch-controlled culture of marine microalgae, especially strains of Thraustochytrium and Schizochytrium.

Jaswir, I. et al. (Jaswir, I., & Monsur, H. A. 2011. Anti-inflammatory compounds of macroalgae origin: A review. Journal of Medicinal Plants Research, 5(33): 7146-7154.) describes that inflammation, which usually takes place in live tissues, is responsible for several deaths and precursor of some deadly diseases, indicating that anti-inflammatory compounds derived from seaweeds are interesting and constitute a promising substitute of the current anti-inflammatory drugs. Macroalgae have pro- and anti-inflammatory compounds, which include sulfated polysaccharides (fucoidans) from brown seaweeds, alkaloids (caulerpin I, II and III) from red and green seaweeds, polyunsaturated fatty acids (docosahexaenoic acid: DHA, eicosapentaenoic acid: EPA, stearidonic acid: SA, and eicosatrienoic acid: ETA), carotenoids (fucoxanthin and astaxanthin), pheophytin A and vidalols A and B.

As an example, there is currently a PPARγ activator medicament in the market, the synthetic drug rosiglitazone, which has been found to have serious adverse effects, so it was withdrawn from the market. Rosiglitazone is a TZD and is an antidiabetic from the class acting as an insulin sensitizer by binding to PPARγ in fat cells (adipocytes), thus, sensitizing adipocytes to insulin, being effective in reducing blood sugar in type II diabetes. After its commercialization, studies were presented that relate it to cardiovascular risk, including risk of heart attack and death, which causing that medical doctors stop prescribing it to their patients in some countries, and in those cases where it is still used, the risks must always be informed to the patients.

Therefore, a naturally occurring nutraceutical, which allows PPARγ to be activated without these side effects of synthetic drugs, such as weight gain due to increased adipose tissue, is one of the aims of the present disclosure. In a preferred embodiment, the disclosure is directed to the neuroprotective action of the extract enriched in free fatty acids and natural PPARγ modulators of the disclosure without some of the side effects, such as weight gain due to increased adipose tissue. In the specific field of neuroprotectives, long-chain omega 3 polyunsaturated fatty acids have been used for their beneficial properties on cardiovascular and neurological health; thus, eicosapentaenoic acid (EPA) and docosahexaenoic acid (DHA) have promoted the use of supplements derived from fish oil in the food industry, pharmaceutical industry and even the cosmetic industry for their beneficial effects altogether. After being ingested, EPA and DHA are rapidly incorporated into the phospholipids of cell membranes, from where these can be released by phospholipases, lipoxygenase and cyclooxygenase enzymes, resulting in products with potent cytoprotective and, especially, anti-inflammatory properties, which would have potential applicability for the prevention or treatment of pathologies such as cardiovascular diseases, neurodegenerative diseases, cancer, inflammatory bowel disease, rheumatoid arthritis and ischemia-reperfusion damage. On the other hand, DHA, for example, has effects on neuronal growth and differentiation, acquiring an important role in neurogenesis and brain development. Also, a neuroprotective role in neurodegenerative diseases, such as Alzheimer's disease and Parkinson's disease by reducing mitochondrial dysfunction, neuroinflammation and oxidative stress has been suggested for DHA.

The publication US2010166918 shows a food supplement formulated as a neuroprotector or promoter of cognitive health, containing a component enriched with activated fatty acids, which corresponds to a modified fatty acid, mainly nitrated fatty acid or keto-fatty acid from an unsaturated fatty acid, which may include palmitic acid, among others, as secondary agent. It is further indicated that it can be useful in cardiovascular events, such as ischemia, wherein the activated fatty acids act as protectors. However, this document does not describe the supplement as obtained from Agarophyton chilensis, or the use of an extract from the said algae containing 8-HETE and 9-HETE and other eicosanoids in combination with palmitic acid, stearic acid, myristic acid, oleic acid, failing to anticipate the disclosure. Moreover, the said document describes that further compounds could be used as secondary agents, which could encompass, for example, antagonists or agonists of several PPARs, including PPARγ. This indicate that the components of the supplement proposed above (publication US2010166918), which is enriched with activated fatty acids, would not have the same activity as the components of the enriched extract in the presently claimed disclosure.

Surprisingly, the inventors have found an extract enriched with naturally occurring free fatty acids, corresponding to a sub-extract obtained by chromatographic separation of an Agarophyton chilensis oleoresin comprising palmitic acid and 8-HETE. In addition, said extract may also contain among its components, stearic acid, myristic acid, oleic acid, and 9-HETE, together with other eicosanoids, wherein said extract has marked neuroprotective effects, and is capable of activating PPARγ without causing adipogenic effects. This enriched extract is useful in the treatment and prevention of neurological, chronic and acute diseases, including ischemia, chronic and acute inflammatory disorders, and also as insulin sensitizing antidiabetic.

### SUMMARY

The disclosure is directed to a fatty acid-enriched extract and to natural modulators of the PPARγreceptor (referred to as Extract B), obtained from the chromatographic separation of an Agarophyton chilensis oleoresin, wherein an extract was obtained enriched with free fatty acids and natural ligands for PPARγ. The resulting extract is enriched with palmitic acid and 8-HETE, and it further comprises stearic acid, myristic acid, oleic acid, and 9-HETE, as well as further oxidized derivatives from arachidonic acid.

The presently claimed extract is intended for the prevention, mitigation, and treatment of chronic or acute neurological diseases, even damage associated with acute stroke, for solving inflammation, and for use as an insulin sensitizer and as a natural activator of PPARγ, which can be used as a nutraceutical extract or as a nutritional component or ingredient.

In an embodiment, the disclosure refers to an Agarophyton chilensis extract enriched with free fatty acids and PPARγ modulators, comprising palmitic acid and 8-HETE, further comprising stearic acid, myristic acid, oleic acid, and 9-HETE. In a preferred embodiment, said enriched extract also comprises:

| | |
|---|---|
| palmitic acid | 12-26% |
| stearic acid | 3.7-4.9% |
| myristic acid | 2.2-5.8% |
| oleic acid | 0.8-6.8% |
| 8-HETE | 0.2-0.5% |
| 9-HETE | 0.017-0.009% |

further eicosanoids

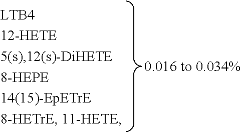

All values indicated above are wt. % (w/w), and have been obtained and statistically validated during the development of the presently claimed disclosure, wherein said acids correspond to LTB4 (Leukotriene B4), 12-HETE (12-Hydroxy-Eicosatetraenoic Acid), 5(s),12(s) -DiHETE (5,12 di-Hydroxy-Eicosatetraenoic Acid), 8-HEPE (8-Hydroxy Eicosapentaenoic Acid), 14(15)-EpETrE (14,15-Epoxy-Eicosatrienoic Acid) 8-HETrE (8-Hydroxy-Eicosatrienoic Acid) and 11-HETE (11-Hydroxy-Eicosatetraenoic Acid).

In another embodiment of the disclosure, said enriched extract of the disclosure, in free fatty acids and PPARγ modulators, optionally comprises further active components, wherein said active components are selected from oils, antioxidants, vitamins and drugs.

In a further embodiment, said extract enriched with free fatty acids and PPARγ modulators optionally comprises Omega 3 oil.

In another embodiment, the disclosure refers to a nutraceutical extract comprising said Agarophyton chilensis extract enriched with free fatty acids and PPARγ modulators, which is obtained from the chromatographic separation of an Agarophyton chilensis oleoresin.

In an embodiment, the disclosure refers to a nutraceutical extract containing low levels of arachidonic acid (AA) and contains less than 0.01% of proinflammatory prostaglandins, wherein AA is comprised between 0.04-0.36%.

In a further embodiment, the disclosure refers to the use of said Agarophyton chilensis extract enriched with free fatty acids and PPARγ modulators, or of said nutraceutical extract for preparing a nutraceutical composition.

Moreover, in an additional embodiment, said use of the nutraceutical extract is useful in the treatment or prevention of chronic and acute neurological diseases; chronic or acute inflammatory disorders; as insulin-sensitizing antidiabetic; stroke; type II diabetes; and hypertension.

In further embodiments, the use of the nutraceutical extract is intended for providing preventive, mitigating and/or therapeutic effects on ischemia or stroke in an individual in need thereof. Said use of the nutraceutical extract is intended for preventing, mitigating and/or treating cerebrovascular disorders mediated by the activation of the PPARγ transcription factor, with the advantage that it does not increase the adipogenesis index in the individual being administered with it.

Furthermore, in an additional embodiment, said Agarophyton chilensis extract enriched with free fatty acids and PPARγ modulators, is used for preparing a nutritional supplement.

In further embodiments, said nutritional supplement can be used as neuroprotector, antidiabetic, or anti-inflammatory agent, or for providing preventing, mitigating and/or therapeutic effects in a patient at risk of suffering or who has already suffered an ischemia or stroke.

The disclosure also refers to the use of the nutritional supplement for the preventing, mitigating and/or therapeutic action of cerebrovascular disorders mediated by the activation of the PPARγtranscription factor.

In an additional embodiment, the disclosure refers to the method of obtaining said Agarophyton chilensis extract enriched with free fatty acids and PPARγ modulators, by means of the following steps:
a) providing an oleoresin obtained by extraction with solvents selected from dichloromethane from the lyophilized and ground biomass of the Agarophyton chilensis algae;
b) extracting by aminopropyl column chromatography said oleoresin dissolved in a load solvent (hexane: diethyl ether: acetic acid (100:3:0.3), and eluting with a solvent mixture A (chloroform: 2-isopropanol, 2:1), to remove the neutral components;
c) eluting from the column of step b) with a solvent mixture B (chloroform: methanol: acetic acid, 100:2:2) to obtain an eluate corresponding to Extract B;
d) drying Extract B protected from light, evaporating with a N2 stream and storing at −80° C.;
e) optionally resuspending Extract B in DMSO 99.9% and storing at inert atmosphere at −80° C.

Wherein said oleoresin is obtained by extraction for the lyophilized and ground biomass of the Agarophyton chilensis algae, as described in WO2014186913, wherein said biomass consists of an Agarophyton chilensis alga that has been cultured, harvested, washed, centrifuged, vacuum packed and frozen at between −20° C. and 80° C., to be subjected to the following extracting steps:
a) thawing the packed alga in cold water, removing the coating, washing with a buffer solution at a pH of 7.4, and centrifuging;
b) chopping the alga from step a) at ambient temperature to obtain 1 to 5 mm pieces, freezing, and maintaining it at −80° C. for at least 24 hours;
c) lyophilizing the alga biomass from step b) and store it in vacuum at between −20° C. and −80° C.;
d) thawing the lyophilized biomass from the former step at ambient temperature and grinding it to a particle size of ≤0,5 mm;
e) suspending the finely ground product from step d) in dichloromethane (CH₂Cl₂), at a ratio of 1:2 to 1:10 of the ground product: CH₂Cl₂ (w:v), packing it under N₂ atmosphere, and sealing the package;
f) incubating while stirring the packages at a temperature of 45-25° C., for 10 to 120 minutes;
g) decanting the mixture, vacuum filtering, retrieving the liquid phase, and optionally extracting once again the solid phase;
h) concentrating the liquid phase from step g) at a temperature between 37-39° C. to obtain a semi-finished oleoresin and resuspending it in cyclohexane,
i) freezing at −80° C. and lyophilizing to obtain Extract A.

### BRIEF DESCRIPTION OF THE DRAWINGS

FIG. 1: It shows a flowchart for the processes for obtaining Extract B from Step 2 to Step 4 from Extract A.

FIG. 2: It shows the Extract B recovery percentage (grams of Extract B per 100 g of Extract A) obtained from 8 independent chromatographic separations, carried out in different months of the year (from April 2017 to February 2018). The dotted line represents the mean recovery value of Extract B.

FIG. 3: It shows a comparison of the PPARγ activation induced by Extract A and by Extract B at the same concentrations (30 and 60 μg/mL). The values correspond to the mean of six determinations of independently produced extracts. *p<0.5 between treatment with Extract A 60 μg/mL and control; **p<0.01 between treatment with Extract B 30 μg/mL and control; ***p<0.001, between treatment with Extract B 60 μg/mL and control (Kruskal-Wallis).

FIG. 4: It shows a comparison of the PPARγ activation induced by 25 μM of FMOC-Leu (FMOC), 25 μM of INT-131 (INT), and 60 μg/mL of Extract B. The values correspond to the mean of six determinations of independently produced extracts. **p<0.01, ***p<0.001 ANOVA Tukey Test.

FIG. 5: It shows the PPARγ transcriptional activation induced by 1 μM of rosiglitazone in PC12 cells. The values correspond to the mean of three determinations of independently produced extracts. ***p<0.001 ANOVA Tukey Test between control and RGZ treatment.

FIG. 6: It shows that the PPARγ transcriptional activation induced by 60 μg/mL of Extract B is inhibited by co-treatment with 10 μM of the specific PPARγ inhibitor, T0070907 (T007), in PC12 cells. The values correspond to the mean of three determinations of independently produced extracts. ***p<0.001 ANOVA Tukey Test between Extract B and Extract B with PPARγ inhibitor.

FIG. 7: It shows the quantification of Oil Red O accumulation after a differentiation assay of 3T3-L1 cells into adipocyte cells, treated with 60 μg/mL of Extract A, with 60 μg/mL of Extract B, 25 μmL of FMOC-Leu (FMOC) or 1 μM of rosiglitazone (RGZ). The values correspond to the mean of three determinations of independently produced extracts. **p<0.01 in RGZ treatments relative to the control (Kruskal-Wallis).

FIG. 8: It shows the expression of mRNA expression of the differentiation gene, fabp4, after the differentiation assay of 3T3-L1 cells into adipocyte cells, treated with 60 μg/mL of Extract A, 60 μg/mL of Extract B, 25 μmL of FMOC-Leu (FMOC) or 1 μM of rosiglitazone (RGZ). The values correspond to the mean of four determinations of independently produced extracts. ***p<0.001 in RGZ treatments relative to the control (Kruskal-Wallis).

FIG. 9: It shows the survival of cortical neurons in culture after damage induced with oligomycin A; wherein 100% corresponds to survival found under basal conditions without treatment with oligomycin. Neurons were treated with Extract B in different concentrations (10-50 μg/mL) or with DMSO (white bar, 0) as carrier, and then they were damaged with oligomycin A. The values correspond to the mean of three independent determinations.

FIG. 10: It shows the survival of cortical neurons in culture after damage induced with oligomycin A; wherein 100% corresponds to survival found under basal conditions without treatment with oligomycin. It shows the comparison of the survival of neurons treated with Extract A or Extract B (10 and 50 μg/mL) after damage with oligomycin. ***p<0.001 in treatments with Extract B in all concentrations relative to the control (0) (ANOVA, Tukey Test).

### DETAILED DESCRIPTION OF THE DISCLOSURE

The disclosure refers to a fatty acid enriched extract with the capability of transcriptionally activating and/or modulating the PPARγ receptor (Extract B) and having neuroprotective and antioxidant activity and further having the advantage of having no adipogenic activity. As described above, the PPARγ pharmacological agonists used are drugs called TZD, which have the disadvantage of increasing adipogenesis or having a capability of favoring cell differentiation into adipocytes.

Said enriched extract is obtained by chromatographic fractioning of an oleoresin from Agarophyton chilensis (Extract A), wherein degradation of oxylipins and antioxidants is minimized. Oxylipins are oxidized lipids derived from free fatty acids produced by phospholipases and lipoxygenases, acting as mediators of physiological responses to tissue stress in plants. The claimed extract comprises as main components: palmitic acid and 8-HETE, and it may also comprise stearic acid, myristic acid, oleic acid, and 9-HETE, together with further eicosanoids at lower ratios.

The claimed extract (Extract B) can be used in the prevention, mitigation or treatment of metabolic diseases, including diabetes, dyslipidemias, and chronic and acute neurological diseases, even ischemia, chronic and acute inflammatory disorders, and as insulin sensitizing antidiabetic agent, in pulmonary hypertension, stroke, or any disease that might be treated with PPARγ agonists, given that the activation of this receptor provides cellular and metabolic benefits which translate into a health benefit.

In a preferred embodiment, this extract is useful for preparing a nutraceutical composition of different types, which can be formulated as a nutraceutical supplement, in the form of tablets, syrups, pills, etc., wherein the supplement comprising said nutraceutical composition and has a neuroprotective, anti-inflammatory and antioxidant activity, especially having therapeutic, preventive or mitigating effects on ischemia or stroke. In particular, the nutraceutical supplement has therapeutic, preventive or mitigating effects on cerebrovascular disorders mediated by the activation of the PPARγ transcriptional factor.

In a second preferred embodiment, the nutraceutical extract is useful for preparing a nutritional supplement, wherein said nutritional supplement has neuroprotective activity, especially therapeutic, preventive or mitigating effects on ischemia or stroke. In particular, the nutritional supplement has therapeutic or preventive effects on cerebrovascular disorders mediated by the activation of the PPARγ transcriptional factor.

Methods

Preparation of the Claimed Extract A

The process for generating the enriched extract from Agarophyton chilensis is described in WO2014186913, which comprises the steps described as follows:

Natural Fresh Algae Collection and Transportation

The algae are collected from the coastal edge in Chile, preferably live and whole algae are collected. Preferably, the red algae (Agarophyton chilensis) are collected and selected manually, with a size being ≥60 cm long, thus preserving the growing algae. The collected and selected material is transported to the site of acclimatization and subsequent cultivation in thermo-insulated containers, with special care to maintain the moisture of the algae, adding enough seawater.

Acclimation and Cultivation of Algae Collected in Pools Under Standardized Conditions

Once the collected and selected algae arrive at destination, biomass is first washed with drinking water, weighted and provided in open culture tanks with a capacity of 4,600 liters, the tanks having aeration and continuous water replacement for their growth in the free-floating mode covering a cultivation area of 6 m². The tanks and biomass are weekly washed with filtered seawater to avoid epiphytic loading and accumulation of organic matter. Said biomass can be maintained under culture conditions from two to twenty weeks before being processed for extraction of lipidic components and obtaining the extract enriched in fatty acids and oxidized fatty acids. Algae collection and culture is not season-dependent, as the enriched extracts produced in different seasons have the same PPARγ activating capability (data not shown).

Biomass Washing and Freezing

The process of extracting components comprises removing biomass from the culture tanks, conveying it to the processing site, maintaining temperature (>20° C.) and moisture. Excess seawater is removed from the algae by centrifugation and is put in vacuum sealed bags for freezing (−20° C.).

Algae Tissue Disruption and Dehydration Through Lyophilization

The algae are thawed in vacuum in cold water, then they are removed from the bag, and washed with a saline buffer solution (NaCl (137 mmol/L), KCl (2.7 mmol/L), Na₂HPO₄.2 H₂O (10 mmol/L), KH₂PO₄ (2.0 mmol/L), pH 7.4), and excess water is removed by centrifugation.

Then, the algae from the former step is (manually or automatically) chopped at ambient temperature (20-24° C.), obtaining 1-5 mm pieces. The chopped algae are put in petri dishes, trays or bags, and maintained in the freezer at −80° C. for at least 24 hours. The previously frozen algae is lyophilized for 12 to 48 hours, preferably 24 hours; at a temperature of −20° C. to −90° C., preferably at −50° C.; at a pressure of 0.1 Pa (0.001 mbar) to 3 Pa (0.03 mbar), preferably at a pressure of 1.4 Pa (0.014 mbar). Once the lyophilized product is obtained, it is stored in vacuum bags at −20° C.

Algae Grinding and Solid-Liquid Extraction of Lipidic Components: (Extract A, Based on the Technique Described in WO2014186913)

The vacuum lyophilized biomass from the former step is allowed to reach ambient temperature (20-24° C.), then it is weighted and ground to a fine powder with a particle size of ≤0,5 mm. For the process of obtaining Extract A, an Erlenmeyer flask (500 ml) is used, which is added with an amount of the finely ground algae, and dichloromethane (CH₂Cl₂) is incorporated, at a ratio of 1/2 to 1/10 by weight of finally ground algae/volume of CH₂Cl₂, preferably 1:3.6 w/v. Each container is exposed to an atmosphere saturated with N₂, and sealed. Immediately, it is incubated with horizontal stirring at a temperature of 45° C. to 25° C. (34° C.); for a term of 2 hours to 10 minutes (30 minutes).

Thereafter, the mixture is decanted and filtered under vacuum in a sintered glass filter and Whatman No. 1 paper. The liquid phase is received in another container. The solid phase is resuspended in the solvent, and the previous extraction procedure is repeated, at least once. The filtered liquid is concentrated in a rotary evaporator at a temperature between 37-39° C., resulting in a semi-finished oleoresin with traces of the solvent, dichloromethane. This product is resuspended in a minimum volume of cyclohexane, quickly frozen at −80° C. and lyophilized for 24 hours, finally obtaining Extract A.

Extract A is stored under saturated atmosphere of inert gas (Ar) in order to avoid oxidation processes, at −20° C.

Obtaining the Oily Extract Enriched With Saturated, Unsaturated and Oxidized Free Fatty Acids, and with Antioxidant Capability (Extract B).

The produced Extract A is subjected to a solid phase chromatography extraction to concentrate saturated, unsaturated free fatty acids, and oxidized fatty acids, to obtain Extract B.

Extract B is obtained by solid phase chromatography separation, using aminopropyl columns, with organic solvent mixtures as mobile phases, through which sequential elutions are performed, wherein in a first step the neutral lipid components are extracted with solvents being less polar, such as eluents, and then the polar lipid components are extracted (rich in free fatty acids) with solvents having a higher polarity.

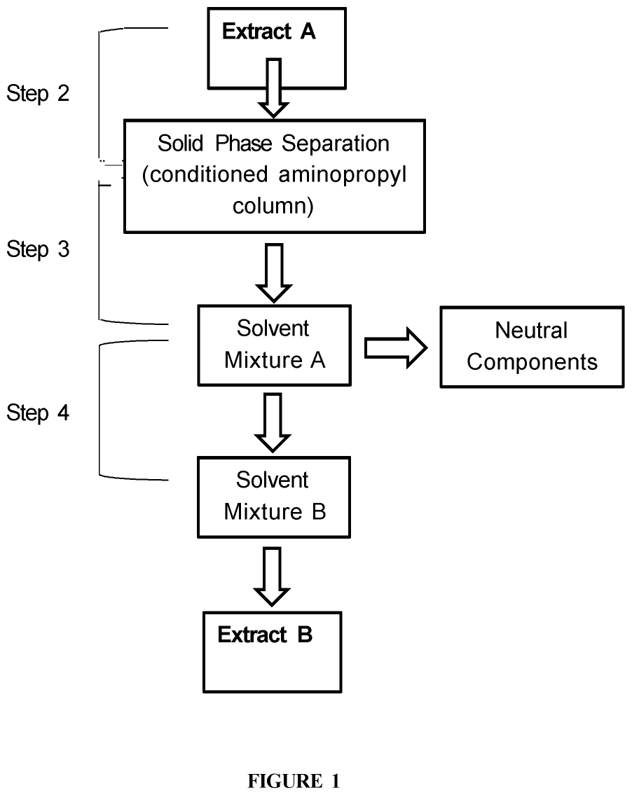

The separation of the sample by the aminopropyl column is carried out with a sample load of 5 wt. % relative to the stationary phase. FIG. 1 shows a scheme for obtaining Extract B from steps 2 to 4. Example 1 describes details about the process for obtaining Extract A and Extract B.

Preparation of the Claimed Extract B

The Steps for Obtaining Extract B are Detailed Next:

The oleoresin obtained by dichloromethane extraction from the lyophilized and ground biomass ofAgarophyton chilensis is provided, which is subjected to the following steps:
a) extracting by aminopropyl column chromatography said oleoresin dissolved in the load solvent, consisting of hexane, diethyl ether, and acetic acid, and eluting with a solvent mixture A, consisting of chloroform and 2-isopropanol, to remove the neutral components;
b) eluting the column from step a) with a solvent mixture B, consisting of chloroform, methanol and acetic acid, to obtain an eluate corresponding to Extract B;
c) drying Extract B protected from light, by evaporation with N₂ stream, and storing at −80° C.;
d) optionally resuspending Extract B in DMSO 99.9% and storing in an inert atmosphere at −80° C.

Wherein the solvent mixtures used in the claimed method are as follows:

| Mixture | Components | Ratio |
|---|---|---|
| Load solvent mixture | hexane:diethyl ether: | 100:3:0.3 |
| | acetic acid | |
| Solvent mixture A | chloroform:2-isopropanol | 2:1 |
| Solvent mixture B | chloroform:methanol: | 100:2:2 |
| | acetic acid | |

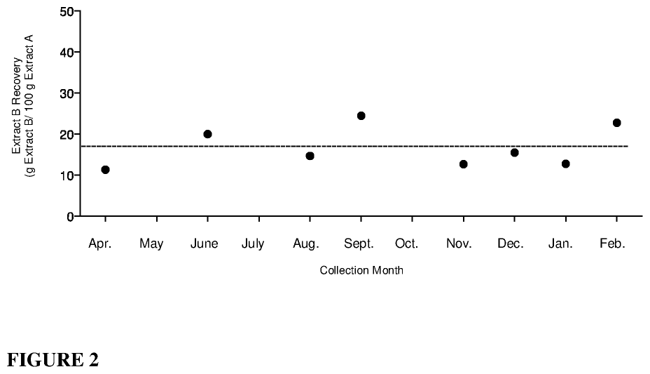

Through the process for obtaining Extract B according to the present disclosure, a product recovery with a yield of 12% to 25% is achieved (FIG. 2).

Optionally, Extract B obtained by chromatographic separation is resuspended in DMSO 99.9% in a concentration of 60 mg/mL and stored in an inert atmosphere at −80° C.

Analysis of Extract A and B components

Characterization of Extract A

During the development of the claimed disclosure, Extract A was assessed to determine the content of antioxidants, such as tocopherols and carotenoids. Additionally, the use of in vitro tests allowed establishing the antioxidant capability of Extract A and Extract B.

Measurements of six different Extracts A show that this Extract has plenty of antioxidant molecules: tocopherols (total: 6673 μg/g of Extract A), the most abundant molecule being γ-tocopherol (5232 μg/g of Extract A) and β-carotene (1538 μg/g of Extract A). The presence of lycopene was not detected in Extract A.

Concerning the content of the different fatty acids present in Extract A, the representative composition shows 51.7% of saturated fatty acids, 18.2% of monounsaturated fatty acids, 24.7% of polyunsaturated fatty acids, and 2.2% of trans fatty acids, palmitic acid being the most abundant fatty acid, with a content of 39%. The full composition is exhibited in Table 1.

TABLE 1
Composition of fatty acids in Extract A of Agarophyton chilensis

| Type | Fatty acid | (%) |
|---|---|---|
| Saturated | Tridecanoic | 1.15 |
| | Myristic | 3.90 |
| | Palmitic | 39.20 |
| | Margaric | 1.30 |
| | Stearic | 4.0 |
| Polyunsaturated | Linoleic | 3.39 |
| | Arachidonic (AA) | 19.40 |
| | Alpha-linolenic (ALA) | 0.61 |
| | Eicosapentaenoic (EPA) | 0.66 |
| | Docosahexaenoic (DHA) | 0.10 |
| Monounsaturated | Oleic | 14.2 |
| Trans | — | 2.25 |

Characterization of Extract B

On the other hand, a lipidomic assay was carried out (Lipidomics Center, Wayne University) to establish the presence of oxidized fatty acid and fatty acid derivatives. Surprisingly, it was found that Extract B is enriched with 8-hydroxy-eicosatetraenoic acid (8-HETE), which is an arachidonic acid (AA) oxidized derivative produced by lipoxygenase, representing 90% of fatty acid oxidized derivatives (Table 2). Moreover, it was determined that per milligram of Extract B, there is a significant presence of 3.5 μg of 8-HETE, 0.1 μg of 9-HETE and 0.25 μg of a mixture of equivalent amount of other arachidonic acid oxidized derivatives, including Leukotriene B4 [LTB4], 12-Hydroxy-Eicosatetraenoic Acid [12-HETE], 5,12 di-Hydroxy-Eicosatetraenoic Acid [5(s), 12(s)-DiHETE], 8-Hydroxy-Eicosapentaenoic Acid [8-HEPE], 14,15-Epoxy-Eicosatrienoic Acid [14(15)-EpETrE], 8-Hydroxy-Eicosatrienoic Acid [8-HETrE], and 11-Hydroxy-Eicosatetraenoic Acid [11-HETE] (Table 2).

TABLE 2
Identification of oxidized fatty acid derivatives present
in the free fatty acid moiety. The content of oxidized fatty acids
(eicosanoids) present in Extract B (n = 3) was assessed.

| | μg/mg |
|---|---|
| Eicosanoid | Extract B |
| 8-HETE | 3.55 |
| 9-HETE | 0.13 |
| LTB4 | 0.06 |
| 12-HETE | 0.05 |
| 5(s), 12(s)-DiHETE | 0.04 |
| 8-HEPE | 0.03 |
| 14(15)-EpETrE | 0.04 |
| 8-HETrE | 0.016 |
| 11-HETE | 0.014 |

Among the fatty acids contained in Extract B, the more abundant ones were found to be palmitic acid, followed by stearic acid, oleic acid and myristic acid (Table 3).

TABLE 3
Profile of the most abundant fatty acids present in Extract B (n = 3).

| | μg/mg |
|---|---|
| Fatty acid | Extract B |
| Palmitic (C16:0) | 191.7 |
| Stearic (Cl8:0) | 42.5 |
| Myristic (Cl4:0) | 39.2 |
| Oleic (C18:l) | 37.6 |
| Dodecanoic (Cl2:0) | 3.8 |
| Lignoceric (C24:0) | 3.7 |
| Palmitoleic (Cl6:1) | 2.0 |
| Linoleic (C16:2w6) | 0.5 |
| Arachidonic (C20:4w6) | 2.5 |
| Eicosapentaenoic (C20:5w3) | 0.09 |

In view of the values described in Tables 1, 2 and 3, it should be noted that in Extract A, the most abundant polyunsaturated fatty acid is AA. In turn, Extract B has a low ratio of this polyunsaturated fatty acid, wherein the content of AA is 0.2% w/w, while Extract A contains 1.9% w/w of AA (Table 1), and it is enriched with its oxidized derivatives 8-HETE and 9-HETE. AA is part of cell membranes, being part of glycerolipids in algae. It is known that AA is a prostaglandin precursor; hence, it has been involved in proinflammatory pathways. Therefore, the biological activities of Extract B could be different and superior to those of Extract A, since the former is not enriched with AA. In addition, as mentioned above, 8-HETE acid acts as natural ligand of PPARs, capable of activating both PPARα and PPARγ.

The extract enriched with free fatty acids and PPARγ modulators ofAgarophyton chilensis of the disclosure contains 19.2% of palmitic acid; 4.2% of stearic acid; 3.9% of myristic acid; 3.8% of oleic acid; 0.35% of 8-HETE; 0.013% of 9-HETE; and 0.025% of further arachidonic acid derivative oxidized fatty acids: LTB4, 12-HETE, 5(s),12(s)-DiHETE, 8-HEPE, 14(15)-EpETrE, 8-HEPE, 8-HETrE, 11-HETE.

It is important to indicate that 20-HETE acid, as well as other hydroxy acids, prostaglandins and leukotrienes were detected in poor abundancy or not detected at all in the analyses (data not shown). In particular, 20-HETE acid has been described as a potent vasoconstrictor involved in the development of hypertension and other pathologies related to inflammation; hence, its absence prevents the use of Extract B from showing undesirable effects associated with said compound. Furthermore, leukotriene B4 was found in low concentrations of 0.006% w/w with a variation between 0.01-0.006%.

Finally, based on the results obtained for the components of the claimed extract, it is possible to state that Extract B, Agarophyton chilensis, contains low amounts of prostaglandins as compared to other related red algae, such as Agarophyton vermiculophylum, which is probably due to a low activity of cyclooxygenase (Honda et al., 2019).

In view of the foregoing, it can be concluded that Extract B is advantageous for use as nutraceutical product, since the low content of AA and derivatives of the same would barely cause undesirable side effects associated with said components.

Furthermore, Extract B derived from Agarophyton chilensis does not have detectable prostaglandins, unlike those that can be detected in a methanolic extract of Agarophyton vermiculophylum (Rempt et al., 2012).

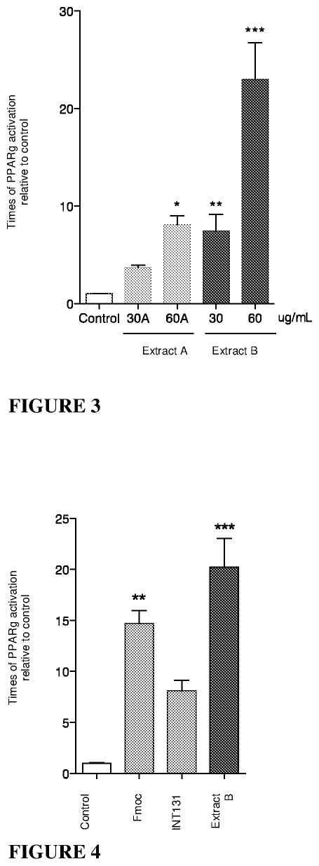

Extract B, compared to Extract A, is enriched with natural ligands of PPARγ, since in PPARγ transcriptional activation assays in PC12 cells, a capability of activating PPARγ is observed in more than twice the activation as compared to the results achieved when using Extract A, as shown in FIG. 3, which exhibits a comparison of the PPARγ transcriptional activation with Extract A and with Extract B in the same concentrations.

The following examples provide the preferred exemplified embodiments of the disclosure, which do not delimit the scope of the disclosure, which can be carried out considering variations related to the details described herein.

In Vitro Assessments and Bioassays

PPARγ Activation Assay in PC12 Cells

The dry Extracts (A and B) were resuspended in DMSO (99.9%) at a maximum dilution of 100 mg/mL, in a preferred dilution range of 10, 20, 25, 30, 35, 40, 45, 50, 55, 60 μg/mL. Dilutions are stored in an inert atmosphere between −20 and 80° C. until the moment of using them in the cellular tests.

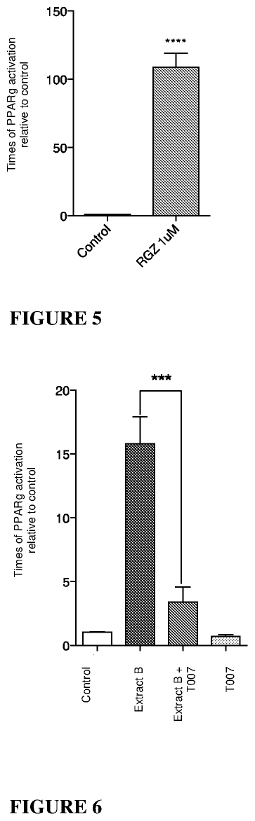

In the PPARγ activation assay, an assay with the reporter gene gal4 PPARγ is performed, which allow assessing the activation of PPARγ induced by ligand, to which end concentrations of 30 and 60 μg/mL were used. PC12 cells were seeded in a 48-well plate at a density of 100,000 cells/well/250 μL of maintenance medium containing the medium RPMI1640® (Roswell Park Memorial Institute-1640 Medium) supplemented with 10% of horse serum (HS), 5% of fetal bovine serum (FBS) and 1× antibiotic/antimicotic (Ab-Am). Then, the DNA (0.8 μg luciferase plasmid (MH100tkLUC), 0.8 μg PPARγGAL4 plasmid and 0.053 μg β-galactosidase plasmid, (β-Gal)) were mixed in OptiMEM® (Opti-Minimun Essential Medium). This constitutes the DNA mixture. In parallel, lipofectamine 2000 was diluted in OptiMEM®. This constitutes the Lipofectamine® mixture. Both mixtures were kept at room temperature for 5 minutes, then were mixed at equal ratios and incubated for 20 minutes at room temperature, while the medium of PC12 cells was replaced with 200 μL of transfection medium (RMPI 1640 supplemented with 2% FBS and 2% HS, without antibiotics) per well. After the 20 min incubation, each well was added with 50 μL of the DNA-lipofectamine mixture. Then, the DNA-lipofectamine mixture was incubated for 6 hours at ambient temperature. Subsequently, the medium of the cells was replaced with a treatment medium (RPMI 1640 supplemented with 2% FBS, 2% HS and 1× Ab-Am). Afterwards, the treatment was carried out with the extracts and its corresponding DMSO control in the treatment medium for 16 hours. In the case of the assays with PPARγ inhibitors, a preincubation for 30 minutes was performed with the PPARγ inhibitor, T0070907 (10 μM), and the inhibitor was maintained for the corresponding 16 hours of treatment (n=3, for each condition). After the preceding incubation, the treatment medium was removed and the cells were lysed with 100 μL of lysis buffer 1× CCLB® (Cell Culture Lysis Buffer, luciferase kit, dilution of stock 5× in water 3×). The lysate was collected, centrifuged for 1 minute at 14000 rpm, the β-galactosidase and luciferase activity in the supernatant was measured, and the times of PPARγ activation was calculated relative to the DMSO control (final concentration in the medium of 0.1%). In parallel, the activity of β-galactosidase was assessed (colorimetry). The luciferase activity values were relativized to the beta-galactosidase activity. The activity results were expressed as times of increase in relation to the activity obtained with control cells (treated only with DMSO). With this assay, both Extract A and Extract B were observed as having PPARγ activators. As shown in FIG. 3, Extract A triggers 3.7 and 7.8 times the receptor activity at 30 μg/mL and 60 μg/mL, respectively (n=6), while Extract B triggers 5.9 and 20.2 times the activity at 30 μg/mL y 60 μg/mL, respectively (n=6), wherein the activity of the last concentration was significantly higher (p<0.01, ANOVA, Tukey post-test) than the one shown by the negative control (cells treated with 0.1% DMSO carrier). This activation level is similar to the level observed with SPPARM-type PPARγ partial activators, such as FMOC-Leu and INT-131 (FIG. 4). Treatment with 25 μM of FMOC-Leu or INT-131 triggered an increase in the PPARγ transcriptional activity in a significantly greater manner as compared to the negative control, being 14.7 and 8.1 times higher (n=10, p<0.01 ANOVA, Tukey post-test), respectively. On the contrary, TZD, rosiglitazone, called a full agonist, at the concentration of 1 μM increased 109 times the PPARγ transcriptional activity (n=9) (FIG. 5). The PPARγ transcriptional activity assay by the PPARγGAL4 system is a highly specific cell assay system. It shows that PPARγGAL4 activation is only mediated by the binding of PPARγ agonists to the nuclear receptor ligand binding domain. This specificity is evidenced when incubating with the PPARγ pharmacological inhibitor, T0070907 (T007). As shown in FIG. 6, simultaneous treatment of PC12 cells transfected with plasmids that allow assessing PPARγ transcriptional activity with Extract B and the inhibitor T0070907 significantly prevented activation induced by Extract B, thereby reducing its activation 3.4 times relative to the control (n=2 in quadruplicate). P<0,01. ANOVA, Tukey post-test.

Induction Assays of cell Differentiation into Adipocytes

In order to assess the effect of the claimed extracts on differentiation of fibroblasts into adipocytes, 3T3-L1 cells are cultured at low passage, avoiding cells from reaching confluence. In order to differentiate 3T3-L1 cells, 600,000 cells were seeded in a 35 mm plate in a 2 mL volume of maintenance medium for staining with Oil-Red-O. In parallel, 90,000 cells per well were seeded in a 250 μL volume of maintenance medium, in a 48-well plate, for a viability assay using tetrazolium salt (MTT), and in a 12-well plate 210,000 cells per well were seeded in a 1 mL volume for extraction of mRNA. Thereafter, the maintenance medium was replaced. It was kept for 48 hours, and then it was replaced to a differentiation medium I (base differentiation medium supplemented with 1 μg/mL insulin, 0.5 mM isobutylmethylxanthine (IBMX), and 0.1 μg/mL dexamethasone) in the absence or presence of the following PPARγ activators: rosiglitazone, RGZ (1 μM), FMOC-Leu (25 μM) or with Extracts A (60 μg/mL) or B (60 μg/mL).

The treatments were incubated for 48 hours, and then were changed to a differentiation medium II (base differentiation medium supplemented with 1 μg/mL insulin) for another 48 hours. Subsequently, they were kept in the base differentiation medium (DMEM supplemented with 10% FBS and 1× penicillin/streptomycin).

On the 7th day of in vitro culture, mRNA was extracted to assess gene expression of differentiated adipocytes, to which end the cultured and differentiated cells were lysed on the 7th day of culture, and mRNA was extracted in HiBind® RNA mini column, according to the manufacturer's instructions. The extraction quality was checked through integrity in gel and any genomic contaminant was removed with the DNAase enzyme. Complementary DNA (cDNA) was generated by reverse transcription of mRNA (RT-MLV and random primer). Then, the resulting cDNA was amplified by qPCR with SRYBRGreen. Three reference genes (GAPDH, ribosomal subunit 18S and b-actin) were simultaneously amplified to quantify expression of the gene of interest encoding FAPB-4 (Fatty-Acid-Binding-Protein 4), which is a protein that increases transcription during adipogenesis.

On the 10th day, staining with Oil Red O (staining for neutral lipids) was performed, to assess lipid (triglyceride) accumulation inside the differentiated cells, which is a feature of the differentiation of fibroblast into adipocytes. To this end, 3T3-L1 cells were washed two times with lx sterile phosphate buffered saline (PBS) at 37° C. Cells were fixed with 4% paraformaldehyde (PFA) 4% in 1× PBS for 1 hour at ambient temperature, and then were washed with PBS and deionized water. Isopropanol 60% was added for 6 minutes, and then were allowed to dry completely. Thereafter, the Oil Red O solution (6:4) was added, 1 ml per 35 mm well, for 2 hours at ambient temperature.

The next day images were captured by light microscopy, using 4× and 10× lenses of cell staining. Then, the isopropanol solubilized staining was quantified by spectrophotometry at 490 nm.

Furthermore, a viability assay with MTT was carried out, wherein cells were washed with PBS and incubated with 0.5 mg/mL of MTT in PBS at 37° C. for 2 hours. After removing MTT, DMSO (99.9%) was added to lyse cells and dissolve MTT/MTT formazan. In order to assess absorbance at 570 nm and 620 nm in a spectrophotometer, 100 μl of this solution were moved to a 96-well plate. The difference between absorbances was calculated, and the survival relative to the control (DMSO) was determined.

The differentiating phenotype is observed in the accumulation of staining for neutral lipids, Red-Oil-O. FIG. 7 shows that both Extract A and Extract B exhibited an accumulation close to that of the control (Extract A: 1.1 times; Extract B: 1.2 times; n=4). It should be noted that these values were close to the values obtained with the treatment with the SPPARM-type agonist FMOC-Leu 25 μM (Fmoc: 1.1; n=4) and that these values are compared to those obtained with the treatment with the full agonist (rosiglitazone), which shows a significant accumulation of neutral lipids compared to the control, more than double in comparison to the control (RGZ: 2.5 times; n=4; p<0.5, Kruskal-Wallis test). The lack of staining with the agonists present in Extracts A and B is not due to the effect on cell viability, since the MTT assay shows that all treatments exhibit a cell survival over 90% (RGZ: 96.5%; FMOC: 100%; Extract A: 96%; Extract B: 92.4%; n=3).

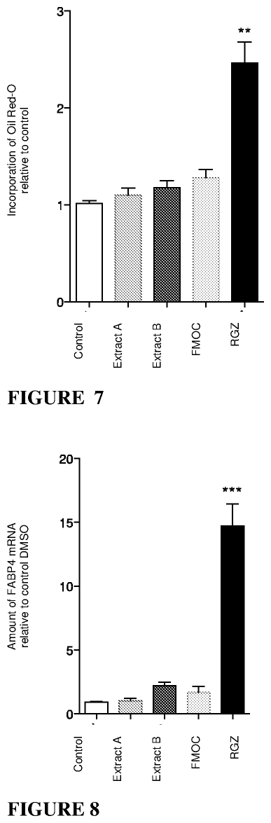

The observation made in view of the staining is correlated to the expression of the adipocyte differentiation marker, the fabp4 gene. As shown in FIG. 8, only those cells treated with the agonist rosiglitazone display a significant induction of this marker (n=3; p<0.5, Kruskal-Wallis test), while the treatment both with the SPPARM-type agonist FMOC-Leu and with Extracts A and B show a low expression of this marker, close to the control DMSO (RGZ: 14.7 times; FMOC: 1.7 times; Extract A: 1.1 times; Extract B: 2.2 times; n=3).

Determining the Antioxidant Capacity of the Extract

The antioxidant capacity of Extracts A and B (resuspended in DMSO) was established with the Oxiselect™ Total Antioxidant Capacity (TAC) kit (Cell Biolabs), in order to assess the antioxidant capacity of lipid samples. In this method, copper is reduced by the samples or standard, where the reduced copper interacts as a chromogen to form a compound that absorbs at 490 nm. As standard, a uric acid solution (serial dilutions) freshly prepared in DMSO 50% in water from a 60 mM stock solution in NaOH 1N, with distilled water, was used.

The Extracts were mixed with the reaction buffer in methanol, according to the manufacturer's instructions. The mixture was incubated for 5 minutes with orbital shaking, then the stopper solution was added, and the reaction was quantified by measuring absorbance in spectrophotometer at 490 nm.

All reactions were produced in duplicate. In order to carry out the comparison to other natural products, the same process described in the preceding sections was performed, to generate an oily extract from commercial maqui and spirulina powder samples. The results were expressed as uric acid equivalents/100 mg of sample.

The antioxidant capacity of the Extracts, which is directly proportional to the increase in absorbance, was expressed as copper reducing equivalents and it was found that 5 mg of Extract A has an antioxidant capacity similar to that obtained with 20 μg/mL (micrograms per milliliters) of pure α-Tocopherol. In turn, the total antioxidant capacity of Extract B is 1.8 times the total antioxidant capacity of oleoresin (Table 4).

TABLE 4
Total antioxidant capacity of Extracts A and B of Agarophyton chilensis

| | mg Uric Acid Eq/100 mg Extract |
|---|---|
| Sample | Mean |
| Extract A | 430 |
| Extract B | 760 |
| Spirulina oily extract | 344 |
| Maqui oily extract | 305 |

Neuroprotection

Neuroprotection Assay

To assess the neuroprotective capacity of Extract B, a model of neuronal damage caused by chemical ischemia was used, preferably the model of damage caused by oligomycin A. Oligomycin A is a macrolide that blocks oxidative phosphorylation, inhibiting mitochondrial ATP synthase.

In order to assess the neuroprotective capacity, a primary culture of cortical neurons of stage E18 rat embryos was used. To this end, adult female rats were acquired, pregnant at stage 18, from the CINBIOT bioterium of the Faculty of Biological Science, Pontificia Universidad Católica de Chile. The rats are maintained at a temperature of 20° C. with water and feed ad libitum under the conditions approved by the ethical committee for the management of laboratory animals, in accordance with the guidelines established by the Chilean Commission for Scientific and Technological Research, CONICYT. Euthanasia was performed by CO₂ inhalation, pursuant to the work protocol, to obtain the cortical neurons. Embryonic brain cortexes were placed in Hank's Balanced Salt Solution (HBSS®) in ice and were incubated with trypsin for 15 minutes at 37° C. for disaggregation, then were washed 3 times with HBSS and Minimum Essential Medium (MEM) was added as adhesion medium with 10% horse serum (HS). The tissue was mechanically disaggregated. Then, 110,000 cells/well were seeded in 48-well plates. The following day (DIV01), the medium was replaced with a maintenance medium (Neurobasal 2% B27®, 1× penicillin/streptomycin). Subsequently, the culture was treated with 1 μM Ara-C (Cytokine (β/δ Arabinofuranoside) to remove the contaminant glia, and each 2 days ⅓ of the medium was replaced with neurobasal medium 2% B27, the cortical neurons were maintained in culture for 8 days, in controlled atmosphere at 37° C., then the neurons were treated with Extract B (10, 25 and 50 μg/mL) dissolved in the culture medium, using DMSO as carrier, and as preservative of the extract, vitamin E was used (10 μM in all concentrations tested).

On the 9th of culture, the challenge with oligomycin A was carried out. Firstly, an incubation at 37° C. with oligomycin A 2 μM was conducted for 30 minutes, then it was changed to a neuronal maintenance medium plus extract. Thereafter, neuronal viability was determined, by removing the entire culture medium, it was washed once with PBS and incubated with 0.5 mg/mL of MTT in neurobasal medium without phenol red at 37° C. for 2 hours to establish the mitochondrial activity, wherein MTT (yellow) is transformed into MTT formazan (dark blue), when reduced by live cells. After removing MTT, pure DMSO was added, to lyse neurons and dissolve MTT/MTT formazan. Subsequently, 100 μl of this solution were moved to a 96-wel plate to measure absorbance at 570 nm and 620 nm in spectrophotometer. The difference between absorbances (570-620) was calculated, and the survival was estimated relative to the control treated with DMSO and without damage caused by oligomycin A. The presence of MTT formazan is quantified at 570 nm. The neuronal survival is expressed as percentage of the control (DMSO).

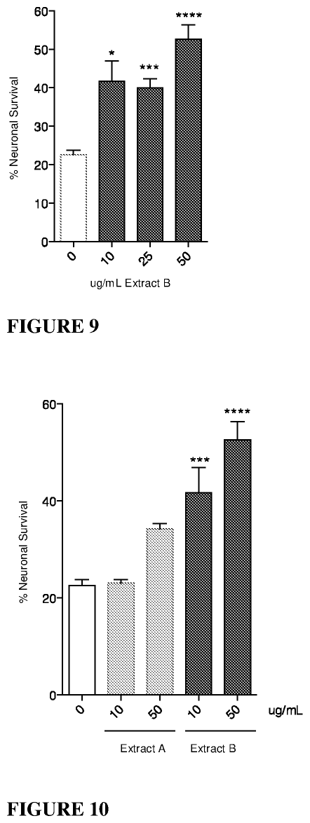

The results obtained from the above-mentioned assay indicated that the treatment with oligomycin A reduced neuronal viability at 22.5% (FIG. 9). It should be noted that the treatment of neurons with Extract B (10, 20 and 50 μg/mL) increases neuronal survival, obtaining a viability between 42-53% relative to the control treated with DMSO 0.1%. Therefore, the treatment with Extract B in all concentrations tested has a statistically significant neuroprotective effect relative to the control (p<0.001 ANOVA, Tukey Post-test). On the other hand, it was verified that the treatment with only vitamin E (10 μM) does not provide protection against oligomycin A (data not shown). Upon comparing the neuronal protection of Extract B to Extract A, it is observed that Extract A has a lower protective effect as the one obtained in the same concentration with the treatment with Extract B (one-way ANOVA, Tukey post hoc; p<0.05; FIG. 10).

This example shows that Extract B has a protective capacity against neuronal damage induced with oligomycin A, indeed the treatment of cortical neurons with Extract B significantly reduced the neuronal death induced by oligomycin A allowing substantial protection of neuronal viability. All together these results indicate that this extract has a surprising greater neuroprotective capacity than Extract A (FIGS. 9 and 10).

This viability assay shows a high reproducibility among assays, even though it is carried out in a primary culture, and due to its characteristics, it shows per se a natural variability.

1. Fraction of an Agarophyton chilensis oleoresin extract-enriched with free fatty acids and PPARγ modulators, and free of neutral lipids, wherein it comprises palmitic acid, stearic acid, myristic acid, oleic acid, and 8-hydroxyeicosatetraenoic acid (8-HETE).

2. The Agarophyton chilensis extract enriched with free fatty acids and PPARγ modulators according to claim 1, wherein it comprises:

| | |
|---|---|
| 12 to 26 wt. % | of palmitic acid; |
| 3.7 to 4.9 wt. % | of stearic acid; |
| 2.2 to 5.8 wt. % | of myristic acid; |
| 0.8 to 6.8 wt. % | of oleic acid; |
| 0.2 to 0.5 wt. % | of 8-HETE. |

3. The Agarophyton chilensis extract enriched with free fatty acids and PPARγ modulators according to claim 2, wherein it further comprises: 0.017 to 0.009% of 9-HETE and 0.016 to 0.034% of the group of further eicosanoids formed by Leukotriene B4, 12-hydroxy-eicosatetraenoic acid, 5,12 di-hydroxy-eicosatetraenoic acid, 8-hydroxy-eicosapentaenoic acid, 14,15-epoxy-eicosatrienoic acid, 8-hydroxy-eicosatrienoic acid, and 11-hydroxy-eicosatetraenoic acid.

4. The Agarophyton chilensis extract enriched with free fatty acids and PPARγ modulators according to any one of claims 1 to 3, wherein it comprises per milligram of extract B 191.7 micrograms of palmitic acid, 42.5 micrograms of stearic acid, 39.2 micrograms of myristic acid, 37.6 micrograms of oleic acid, 3.5 micrograms of 8-HETE, 1.3 micrograms of 9-HETE, and 0.25 micrograms of further eicosanoids.

5. Method of obtaining the extract enriched with free fatty acids and PPARγ modulators according to claim 1, wherein an oleoresin obtained by dichloromethane extraction from the lyophilized and ground biomass of the Agarophyton chilensis algae, wherein that the method comprises the steps of:
a) providing an oleoresin extracted with dichloromethane from the lyophilized and ground biomass of the Agarophyton chilensis algae;
b) extracting by aminopropyl column chromatography said oleoresin dissolved in a load solvent (hexane, diethyl ether, acetic acid), and eluting with a solvent mixture A (chloroform: 2-isopropanol, 2:1), to remove the neutral components;
c) eluting from the column of step b) with a solvent mixture B (chloroform, methanol, acetic acid) to obtain an eluate or Extract B;
d) drying Extract B protected from light, evaporating with a N₂ stream and storing at −80° C.;
e) optionally resuspending Extract B in DMSO 99.9% and storing at inert atmosphere at −80° C.

6. The method according to claim 5, wherein the load solvent mixture contains hexane:
diethyl ether: acetic acid in a ratio of 100:3:0.3.

7. The method according to claim 5, wherein the solvent mixture A contains chloroform: 2-isopropanol in a ratio of 2:1.

8. The method according to claim 5, wherein the solvent mixture B contains chloroform, methanol and acetic acid in a ratio of 100:2:2.

9. Nutraceutical composition, wherein it comprises the Agarophyton chilensis extract enriched with free fatty acids and PPARγ modulators according to claim 1.

10. The nutraceutical composition according to claim 9, wherein it further comprises further active components.

11. The nutraceutical composition according to claim 10, wherein the active components are selected from oils, antioxidants, vitamins and drugs.

12. The nutraceutical composition according to claim 10, wherein it further comprises Omega 3 oil.

13. Use of the Agarophyton chilensis extract enriched with free fatty acids and PPARγ modulators according to claim 1, wherein it is for preparing a nutraceutical composition useful for the treatment or prevention of health problems requiring neuroprotection, wherein said neuroprotection involves activation of PPARγ receptors.

14. The use according to claim 13, wherein the nutraceutical composition is useful for the treatment or prevention of chronic or acute neurological diseases; chronic or acute inflammatory disorders; stroke.

15. The use according to claim 13, wherein the nutraceutical composition is useful for preventing, mitigating and/or treating ischemia or stroke.

16. The use according to claim 13, wherein the nutraceutical composition does not cause an increase in adipogenesis.

17. The use of the enriched extract according to claim 1, wherein it is for preparing a nutritional supplement, useful as neuroprotector.

18. The use according to claim 18, wherein the nutraceutical supplement is useful for preventing, mitigating and/or treating ischemia or stroke.
